(19) United States
(12) Patent Application Publication
(10) Pub. No.: US 2022/0145267 A1
(43) Pub. Date: May 12, 2022

(54) METHOD FOR PRODUCING CULTURED CELLS

(71) Applicants: FUKUSHIMA MEDICAL UNIVERSITY, Fukushima-shi, Fukushima (JP); ZENOGEN PHARMA CO.,LTD., Karlyama-shi, Fukushima (JP)

(72) Inventors: Yuji YOKOUCHI, Fukushime-shi, Fukushima (JP); Takumi ERA, Fukushime-shi, Fukushima (JP); Shinichi SUZUKI, Fukushima-shi, Fukushima (JP)

(73) Assignees: FUKUSHIMA MEDICAL UNIVERSITY, Fukushima-shi, Fukushima (JP); ZENOGEN PHARMA CO.,LTD., Karlyama-shi, Fukushima (JP)

(21) Appl. No.: 17/436,701

(22) PCT Filed: Mar. 6, 2020
(86) PCT No.: PCT/JP2020/009554

§ 371 Date: Sep. 7, 2021

(30) Foreign Application Priority Data
Mar. 8, 2019 (JP) 2019-042383

(51) Int. Cl. C12N 5/10; C12N 5/04; A61K 35/12; C12Q 1/6883; C12N 15/85

(52) U.S. Cl. CPC C12N 5/10; C12N 5/04; C12N 15/85; C12Q 1/6883; A61K 35/12

(57) ABSTRACT

In one embodiment, the present invention provides a method for a plurality of cells comprising screening of a part of the cells without subculturing all of the cells and selecting a part of the cells based on the results.

In one embodiment, the present invention relates to a method of producing a culture cell, comprising the steps of: a) culturing a plurality of animal cells or plant cells in a culture vessel by adherent culture or on a semisolid medium to form a plurality of colonies each of which is derived from a single cell; b) detecting a sequence of one or more base(s) in a nucleic acid for a part of said plurality of colonies by a nucleic acid or protein detection method, during which said plurality of colonies are cultured in a culture vessel; and c) selecting and collecting said part of the colonies based on the results of said detection method.

### TECHNICAL FIELD

In one embodiment, the present invention relates to a method of producing a culture cell.

### BACKGROUND ART

It is estimated that the human genome has 10⁷ single-nucleotide polymorphisms (SNPs), and it is considered that these SNPs determine the human phenotypes such as the form, constitution, and predisposition. In particular, a SNP related to a disease is called a pathogenic SNP, and is known to directly or indirectly cause hereditary diseases, for example, multiple endocrine neoplasia type 2 (MEN2B) or dystrophic epidermolysis bullosa (DEB) (Non-Patent Literature 1 and 2).

Disease-specific induced pluripotent stem cells (iPSCs) having a pathogenic SNP or a genetic mutation have been produced from patients, and used as an in vitro model disease (Non-Patent Literature 3 and 4). Repairing these iPSCs to produce isogenic revertant cells is a promising strategy for genome editing, and also can help to develop a novel therapy (Non-Patent Literature 4). However, genome editing procedures are complicated at present, and an application thereof requires an accurate and simple genome editing method.

### CITATION LIST

### Non-Patent Literature

Non-Patent Literature 1: Wellcome Trust Case Control Consortium, 2007, Nature, 447, 661-678
Non-Patent Literature 2: Wells, S. A., Jr. et al., 2013, J. Clin. Endocrinol. Metab., 98, 3149-3164
Non-Patent Literature 3: Peitz, M. et al., 2013, Curr. Mol. Med., 13, 832-841
Non-Patent Literature 4: Sanchez-Danes, A. et al, 2012, EMBO Mol. Med., 4, 380-395

### SUMMARY OF INVENTION

### Technical Problem

As described above, genome editing procedures are complicated at present. For example, it is common that selecting genome-edited cells from a plurality of cells conventionally involves subculturing each of all the cells during screening for detection of the genome-edited cells, for example, for the purpose of preventing contamination between the cells. Then, the genome-edited cells are selected from the subcultured cells on the basis of the screening results (for example, see Kwart, D. et al., Nat. Protoc., 12, 329-354). These procedures involve subculturing all the cells subjected to screening, thus resulting in a heavy workload and complication.

Further, genome editing often comprises introducing a base serving as a marker in addition to a base of interest to be edited, in order that subsequent screening can be simplified. A marker base to be used generally comprises one to several bases that cause a silent mutation having no influence on the amino acid sequence. However, a silent mutation can influence the expression efficiency of a protein. In addition, a mutation in the untranslated region is related to transcriptional regulation, splicing control, and the like. Accordingly, it is preferable to introduce no marker base in order to reduce the risk of causing a side effect. In such a case where no marker base is introduced, however, detecting intended genome-edited cells necessitates a step of detecting the absence of a nucleotide existing before substitution by genome editing (negative screening), but this is considered difficult in general.

In one embodiment, the present invention provides a method for a plurality of cells comprising screening of a part of the cells without subculturing all of the cells, and selecting a part of the cells based on the results. In another embodiment, the present invention provides a method that is capable of selecting genome-edited cells without introducing a marker base.

### Solution to Problem

The present inventors have surprisingly found that when a plurality of animal cells or plant cells are cultured and a part of the cells are subjected to a nucleic acid or protein detection method, during which the cells are cultured, a part of the cells can be selected based on the results without contamination. In addition, the present inventors have found that it is possible to select a genome-edited cell with sufficient efficiency without introducing a marker base through negative screening which was previously considered difficult.

The present invention encompasses the following embodiments.

(1) A method of producing a culture cell, comprising the steps of:

a) culturing a plurality of animal cells or plant cells in a culture vessel by adherent culture or on a semisolid medium to form a plurality of colonies each of which is derived from a single cell;

b) detecting a sequence of one or more base(s) in a nucleic acid for a part of said plurality of colonies by a nucleic acid or protein detection method, during which said plurality of colonies are cultured in a culture vessel; and

c) selecting and collecting said part of the colonies based on the results of said detection method.

(2) The method according to (1), comprising a step of further culturing the cells collected in said step c).

(3) The method according to (1) or (2), wherein said gene or protein detection method is single-base mismatch detection PCR.

(4) The method according to any one of (1) to (3), wherein the cells are cultured by adherent culture in said step a).

(5) The method according to any one of (1) to (4), wherein said plurality of colonies are cultured for 4 hours to 6 days in said step b).

(6) The method according to any one of (1) to (5), wherein animal cells are cultured in said step a).

(7) The method according to (6), wherein said animal cells are mammalian cells.

(8) The method according to (7), wherein said mammalian cells are stem cells.

(9) The method according to any one of (1) to (8), wherein said culture vessel has an identifier whereby the position of each colony in the vessel can be identified, and wherein said part of the colonies are identified and selected based on said identifier in said step c).

(10) The method according to (9), wherein said culture vessel having an identifier is a plate with grid.

(11) The method according to any one of (1) to (10), comprising a step of further subjecting said part of the plurality of colonies to another gene or protein detection method before or after said step c).

(12) The method according to any one of (1) to (11), further comprising a step of performing genome editing for said animal cells or plant cells before said step a).

(13) The method according to (12), wherein said genome editing substitutes a mutant base sequence with a wild-type base sequence, or a wild-type base sequence with a mutant base sequence in the genomic DNA.

(14) The method according to (13), wherein said mutant base sequence causes a disease.

(15) The method according to any one of (12) to (14), wherein said genome editing does not introduce a marker base, and wherein the absence of a nucleotide existing before substitution by the genome editing is detected in said step b).

(16) The method according to any one of (12) to (15), wherein said genome editing is performed using a protein selected from the group consisting of TALEN and a modified protein thereof, SpCas9 and a modified protein thereof, SaCas9 and a modified protein thereof, ScCas9 and a modified protein thereof, AsCpf1 and a modified protein thereof, LbCpf1 and a modified protein thereof, FnCpf1 and a modified protein thereof, MbCpf1 and a modified protein thereof, CBE and a modified protein or an analogue thereof, and ABE and a modified protein or an analogue thereof.

(17) A method of producing a culture cell, comprising the steps of:
substituting a mutant base sequence with a wild-type base sequence or substituting a wild-type base sequence with a mutant base sequence in the genomic DNA in an animal cell or plant cell by genome editing without introducing a marker base;
detecting the absence of a nucleotide existing before substitution by the genome editing in the genome-edited cell; and
selecting and collecting a cell in which the absence of the nucleotide existing before substitution is detected.

(18) The method according to (17), wherein a single-base mismatch detection PCR detects the absence of the nucleotide existing before substitution by said genome editing.

(19) The method according to (17) or (18), wherein said genome editing is performed using a protein selected from the group consisting of TALEN and a modified protein thereof, SpCas9 and a modified protein thereof, SaCas9 and a modified protein thereof, ScCas9 and a modified protein thereof, AsCpf1 and a modified protein thereof, LbCpf1 and a modified protein thereof, FnCpf1 and a modified protein thereof, MbCpf1 and a modified protein thereof, CBE and a modified protein or an analogue thereof, and ABE and a modified protein or an analogue thereof.

(20) A genome-edited cell obtained by the method according to any one of (12) to (19).

(21) A pharmaceutical composition comprising the cell according to (20).

The present specification encompasses the contents disclosed in Japanese Patent Application No. 2019-042383, to which the present application claims priority.

### Advantageous Effects of Invention

In one embodiment, provided is a method for a plurality of cells comprising screening of a part of the cells without subculturing all of the cells and selecting a part of the cells based on the results. In another embodiment, the present invention provides a method that is capable of selecting a genome-edited cell without introducing a marker base.

### BRIEF DESCRIPTION OF DRAWINGS

FIG. 1 shows a schematic of a master plate used in Examples. The master plate has a plurality of sections divided by grid so that clones can be identified during use in the first or secondary screening described below and during subsequent collection (in the figure, the sites used for the screening are each marked with ∘; and Figure TA shows the master plate, and FIG. 1B shows a map prepared therefrom). In the MAP shown in FIG. 1B, colonies are marked and numbered.

FIG. 2 shows the results obtained by substituting a wild-type base at the RET_M918 site in a wild-type (WT) allele of FB4-14 MEN2B-iPSC. In FIG. 2A, the first line shows a wild-type allele sequence, the second line shows an ssODN modified template (ssODN_RET_M918T_I1913_silentC (Mut)) having both a modified base at M918 and a marker base at I913, the third line shows a wild-type allele sequence after modification, and the fourth line shows a mutant allele sequence. The right-pointing arrow shows a primer for detecting a marker base. FIG. 2B shows the result of single-nucleotide mismatch detection PCR analysis (the arrowhead shows an amplification product based on a primer sequence for detecting a marker base). FIG. 2C shows the results of direct sequencing of a target sequence. In FIG. 2C, arrows show a substitution of T with C, which causes a substitution of Met with Thr at Met918, and the arrowhead shows a marker base (a substitution of T with C, which causes a silent mutation at Ile913).

FIG. 3 shows the results obtained by performing allele-specific single-nucleotide repair for a pathogenic mutation in an FB4-14 cell, using a repair template having a marker base which causes a silent mutation at Ile913. In FIG. 3A, the first line shows a mutant allele sequence, the second line shows an ssODN repair template (ssODN_RET_M918_I913_silentC (WT)) comprising a repair base at Met918 and a marker base at Ile913, the third line shows a mutant allele sequence after repair, and the fourth line shows an RET wild-type allele sequence. The right-pointing arrow shows a primer for detecting a marker base. FIG. 3B shows the result of single-base mismatch detection PCR analysis (the arrowhead shows an amplification product based on a primer sequence for detecting a marker base). FIG. 3C shows the results of direct sequencing of a target sequence. In FIG. 3C, arrows show a substitution of a mutant base with a wild-type base (a substitution of C with T) at the target site, and the arrowhead shows a marker base (a substitution of T with C, which causes a silent mutation at Ile913).

FIG. 4 shows the results obtained by performing allele-specific single-nucleotide repair for a pathogenic mutation in an FB4-14 cell, using a repair template having a marker base which causes a silent mutation at Ile920. In FIG. 4A, the first line shows a mutant allele sequence, the second line shows an ssODN repair template (ssODN_RET_M918_I920_silentC (WT)) comprising a repair base at Met918 and a marker base at Ile920, and the third line shows a wild-type (WT) allele sequence. The right-pointing arrow shows a primer for detecting a marker base. FIG. 3B shows the result of SNP-PCR analysis (the arrowhead shows an amplification product based on a primer sequence for detecting a marker base). FIG. 4C shows the results of direct sequencing of a target sequence. In FIG. 4C, arrows show a substitution of a mutant base with a wild-type base (a substitution of C with T) at the target site, and the arrowhead shows a marker base (a substitution of T with C, which causes a silent mutation at Ile920).

FIG. 5 shows the results obtained by performing allele-specific single-nucleotide repair for a pathogenic mutation without using a marker base, and FIG. 5A is a schematic diagram thereof. In FIG. 5B, the first line shows a mutant allele sequence, the second line shows an ssODN repair template (ssODN_RET_M918 (WT)) comprising a repair base at Met918, the third line shows a mutant allele sequence after repair, and the fourth line shows a wild-type (WT) allele sequence. The right-pointing arrow represents a primer for detecting a mutant allele. FIG. 5C shows the result of single-base mismatch detection PCR analysis (the arrowheads show that no amplification product based on a primer sequence for detecting a mutant allele is produced). FIG. 5D shows the results of direct sequencing of a target sequence. In FIG. 5D, arrows show a substitution with a wild-type base (a substitution of C with T) at the target site.

### DESCRIPTION OF EMBODIMENTS

1. Method of Producing a Culture Cell

In one embodiment, the present invention relates to a method of producing a culture cell. A cell that can be subjected to the method of the present invention is not limited provided that it is an animal cell or a plant cell. Examples of biological species from which an animal cell is derived include a mammal (for example, a primate such as a human and rhesus monkey; a laboratory animal such as a rat, mouse, and brown rat; a livestock animal such as a pig, cattle, horse, sheep, and goat; a pet animal such as a dog and cat; a marsupial such as a kangaroo, koala, and wombat; and a monotreme such as a platypus and spiny anteater), a bird (a fowl, duck, pigeon, ostrich, emu, parrot, red jungle fowl, and the like), a reptile (a lizard, alligator, snake, turtle, and the like), an amphibian (Xenopus laevis, Xenopus tropicalis, a salamander, axolotl, the like), a fish (a medaka, zebrafish, goldfish, Carassius, carp, salmon, trout, eel, catfish, perch, sea bream, flounders, tuna, yellowtail, skipjack, shark, and the like), a mollusk (an octopus, squid, abalone, turban shell, pearl oyster, clam, Japanese little neck, snail, and the like), an echinoderm (a sea urchin, sea cucumber, starfish, and the like), a crustacean (a crab, shrimp, squilla, crayfish, hermit crab, and the like), an insect (a silkworm, Bombyx mandarina, Eumeta japonica, Eumeta minuscula, fruit fly, honeybee, bumble bee, ant, ladybird, cricket, locust, beetle, stag beetle, longicorn, cockroach, termite, and the like), and a cnidarian (hydra, jellyfish, coral, and the like). A mammal such as a human is preferable. Biological species from which a plant cell is derived may be, but is not limited to, any plant cell, for example, an angiosperm including monocotyledon and dicotyledon, gymnosperm, bryophyte, pteridophyte, herbaceous plant, woody plant, and the like. Specific examples of species from which a plant cell is derived include Poaceae (rice, wheat, barley, maize, Sorghum, pigeon wheat, sugarcane, Phragmites and Phyllostachys, and the like), Brassicaceae (Arabidopsis thaliana, rapeseeds, broccoli, horse radish, and cabbage, and the like), Solanaceae (eggplant, tomato, tobacco, chili pepper, and potato, and the like), Cucurbitaceae (cucumber, melon, watermelon, gourd, and the like), Amaryllidaceae (Allium, onion, garlic, and the like), Leguminosae (soybean, azuki, astragali, licorice, kudzu vine, senna, Astragalus membranaceus, Acacia senegal, red sandalwood, and the like), Ranunculaceae (Coptis japonica, and the like), Rubiaceae (gardenia, coffee tree, and the like), Araceae (taro, Amorphophallus konjac, Rhizoma pinelliae, Pinellia ternata, and the like), Dioscoreacea (Dioscorea japonica, Dioscorea batatas, and the like), Convolvulaceae (sweet potato, morning glory, and the like), Euphorbiaceae (cassava, Hevea brasiliensis, and the like), Umbelliferae (carrot, celery, Angelica acutiloba, Bupleurum falcatum, Cnidium officinale, Siler divaricatum, and the like), Polygonaceae (Polygonum longisetum, buckwheat, rhubarb, and the like), Moraceae (mulberry, paper mulberry, and the like), Amaranthaceae (amaranth, celosia, and the like), Portulacaceae (purslane, rose moss, and the like), Malvaceae (okra, hibiscuses, and the like), Asteraceae (sunflower, Helianthus tuberosus, artichoke, Atractylodes lancea, Atractylodes ovata, and the like), Comaceae (Cornus officinalis, and the like), Rosaceae (rose, peach, pear, apple, strawberry, and the like), Rutaceae (orange, yuzu, Zanthoxylum piperitum, Phellodendron japonicum, and the like), Vitaceae (grape, Vitis coignetiae, and the like), Paeoniaceae (tree peony, peony, and the like), Scrophulariaceae (Rehmannia glutinosa, and the like), Labiatae (perilla, Mentha, rosemary, Scutellaria baicalensis, and the like), Oleaceae (forsythia, and the like), Campanulaceae (balloon flower, and the like), Actinidiaceae (Actinidia polygama, Actinidia arguta, kiwi fruit, and the like), Alismataceae (Alisma plantago-aquatica var. orientale, and the like), Dwarf lilyturf (Dwarf lilyturf, and the like), Ebenaceae (Diospyros kaki, ebony, and the like), Orchidaceae (Cymbidium goeringii, Vanilla, and the like), Musaceae (Musa basjoo, banana, and the like), Araliaceae (Aralia elata, Panax ginseng, and the like), Lauraceae (cassia cinnamon, avocado, and the like), Rhamnaceae (jujube, and the like), Fagaceae (beech, oak, chestnut, and the like), Sapindaceae (Sapindus mukorossi, Japanese horse chestnut, and the like), Cannabaceae (Cannabis sativa (hemp), and the like), Urticaceae (Boehmeria nivea (ramy), and the like), Asparagaceae (sisal hemp, agave, and the like), Anacardiaceae (Rhus trichocarpa, Rhus succedanea, mango, and the like), Betulaceae (Betula, Betula ermani, and the like), Salicaceae (pussy willow, weeping willow, and the like), Piperaceae (pepper, and the like), Bromeliaceae (pineapple, and the like), Caricaceae (papaya, and the like), Myristicaceae (nutmeg, and the like), Papaveraceae (poppy, and the like), Palmae (coconut palm, oil palm, and the like), Pinaceae (Japanese red pine, Picea jezoensis, and the like), Ephedraceae (Ephedra sinica), Ginkgoaceae (ginkgo, and the like), pteridophyte (bracken, field horsetail, tree fem, and the like), and bryophyte (liverwort, hornwort, Bryopsida, hair-cap moss, and the like). These cells may be any of a primary culture cell, a subcultured cell, and a frozen cell.

A cell subjected to the method of the present invention may be, for example, a mammalian cell such as a stem cell. As used herein, a “stem cell” refers to a cell having both the ability to differentiate into another type of cell or various types of cell and the ability to self-renew. A stem cell may be a cell population consisting only of stem cells, or may be a cell population comprising stem cells abundantly. Examples of stem cells of a mammal include undifferentiated cell existing in a living tissue such as bone marrow, blood, skin, intestine, nerve, and fat (collectively referred to as a somatic stem cell, examples of which include a Muse cell), embryonic stem cell (ES cell), induced pluripotent stem cell (iPS cell), and the like. Such a stem cell can be produced by a known methodper se, available from a prescribed institution, or can be purchased in the form of a commercially available product.

In one embodiment, a method according to the present invention comprises the steps of.
a) culturing a plurality of animal cells or plant cells in a culture vessel by adherent culture or on a semisolid medium to form a plurality of colonies each of which is derived from a single cell (hereinafter also referred to as a “colony-forming step”);
b) detecting a sequence of one or more base(s) in a nucleic acid for a part of said plurality of colonies by a nucleic acid or protein detection method, during which said plurality of colonies are cultured in a culture vessel (hereinafter referred to as a “detecting step”); and
c) selecting and collecting said part of the colonies based on the results of said detection method (hereinafter referred to as a “selecting step”). Each step constituting a method according to the present invention in the present embodiment is described in detail below.

a) Colony-Forming Step

In the colony-forming step, a plurality of animal cells or plant cells are cultured by adherent culture or on a semisolid medium in a culture vessel to form a plurality of colonies each of which is derived from a single cell.

The range of “a plurality of” as used herein may be, but is not limited to, for example, 2 or more, 3 or more, 4 or more, 5 or more, 10 or more, 10² or more, 5×10² or more, or 10³ or more, and in addition, may be 10⁵ or less, 10⁴ or less, or 10³ or less. For example, “a plurality of” may be 10² to 10⁴ or 5×10² to 10³. In the colony-forming step, culturing too many cells increases the risk of causing the colonies formed to come in contact with each other, and conversely, culturing too few cells decreases the number of colonies to be subjected to the subsequent detecting step. Considering these factors, a person skilled in the art can suitably select the number of cells. For example, in the colony-forming step, the culture can be performed at a lower concentration (for example, approximately 1 cell/cm² to approximately 100 cells/cm², approximately 5 cells/cm² to approximately 40 cells/cm², or approximately 10 cells/cm² to approximately 20 cells/cm²) than a usual subculture.

The culture conditions (temperature, period, and the like) for the colony-forming step can be selected in accordance with the cell type to be used. For example, the culture temperature for animal cells can be approximately 20° C. to approximately 40° C., approximately 30° C. to approximately 40° C., approximately 35° C. to approximately 39° C., approximately 36° C. to approximately 38° C., or approximately 37° C., and the culture temperature for plant cells can be approximately 10° C. to approximately 30° C., approximately 20° C. to approximately 27° C., or approximately 25° C. The animal cells may be cultured in the presence of CO₂, and the CO₂ concentration may be approximately 2% to approximately 10%, approximately 4% to approximately 6%, or approximately 5%. The culture period for the colony-forming step may be, for example, 4 days to 12 days, 6 days to 10 days, or 7 days to 8 days.

The culture medium to be used in the colony-forming step can be selected in accordance with the cell type to be used. The culture can be performed using a commercially available culture medium (examples of media for animal cells include DMEM, MEM, BME, RPMI 1640, F-10, F-12, DMEM-F12, α-MEM, IMDM, MacCoy's 5A culture medium, or mTeSR1 culture medium; and examples of media for plant cells include Murashige Skoog (MS) culture medium, Gamborg's B5 culture medium, modified Gamborg's B5 culture medium, Linsmaier & Skoog (LS) culture medium) or using a prepared culture medium. These culture media can be supplemented with various additives (for example, a serum or serum substitute, L-glutamine, non-essential amino acid, 2-mercaptoethanol, antibiotics such as penicillin or streptomycin, and growth factor such as a basic fibroblast growth factor).

In the colony-forming step, the culture is performed by adherent culture, or the culture is performed on a semisolid medium (or in a culture medium). The “adherent culture” refers to culturing cells in a culture medium with the cells adhered to the contact surface of a culture vessel. The strength of “adhesion” may be such strength that cells maintaining survivability cannot be released as they are, for example, if not by an artificial treatment such as a tapping treatment, pipetting treatment, or enzyme treatment. To augment the adhesiveness in adherent culture, it is possible to use a culture vessel coated with, for example, an extracellular matrix (for example, laminin, tenascin, fibronectin, collagen, vitronectin or a derivative thereof (such as VT N-N)), Matrigel or a derivative thereof (such as Matrigel-GFR), poly-D-lysine, or the like. As used herein, a “semisolid medium” refers to a culture medium that is neither liquid nor solid and that contains a gelling agent such as agar, agarose, gelatin, collagen, Matrigel or a derivative thereof (such as Matrigel-GFR), fibroin, chitin, chitosan, carrageenan, sodium carboxymethylcellulose, methyl cellulose, xanthan gum, guar gum, pectin, or polyvinyl alcohol. The semisolid medium may have viscosity sufficient to prevent cells added thereto from sinking therein and from coming in contact with and sticking to the inner surface of a container (vessel) containing the semisolid medium placed therein. A semisolid medium can be prepared, for example, by adding a gelling agent in an amount of 0.10% to 5% (w/v) to a liquid culture medium.

Suitably selecting the above-mentioned number, culture period, and culture conditions of cells makes it possible to form a plurality of colonies each of which is derived from a single cell. In this regard, a “colony derived from a single cell” as used herein may be a colony derived from a single cell only, or may be a colony the majority (for example, 50% or more, 80% or more, 90% or more, or 95% or more) of which is derived from a single cell, and which contains a small number of contaminant cells, provided that the effects of the present invention can be achieved.

As used herein, a “culture vessel” is not limited provided that it is used for culturing cells, and examples thereof include a cell culture dish, a cell culture bottle (or flask), a multi-well plate, a microcarrier, and the like. A commercially available culture vessel may be used. Examples of the material of the culture vessel include, but are not limited particularly to, glass, plastic, and the like.

In one embodiment, the culture vessel has an identifier capable of identifying the position of each colony in the vessel in order that a colony from which a base sequence of interest is detected in the detecting step described below can be selected in the selecting step described below. The shape or the like of the identifier may be, but is not limited to, the following: a letter, number, figure such as polygon, arrow, line, dot, marker, and a combination thereof, and may be, for example, a grid (a grid line). The identifier may be directly given to the bottom or the like of the culture vessel, or a sheet, for example, a translucent seal having an identifier may be attached to the bottom or the like of the culture vessel. In addition, a secondary identifier (for example, a marker, check, or the like) to be given on the basis of such an identifier can be used to identify each colony.

b) Detecting Step

The detecting step is a step of detecting, by a nucleic acid or protein detection method, a sequence of one or more base(s) in a nucleic acid for a part of a plurality of colonies formed in the colony-forming step. A “part” of colonies is not limited provided that it is possible to detect a cell of interest by the detecting step, and for example, may be 5% or more, 6% or more, 8% or more, 10% or more, or 20% or more of all the colonies formed, may be 80% or less, 60% or less, 50% or less, 40% or less, or 30% or less, and may be, for example, 5% to 80%, 8% to 40%, or 10% to 30%.

In this step, a part of the colonies is subjected to the detecting step, and, out of the part of the colonies, a colony/colonies from which the above-mentioned sequence of one or more base(s) is detected is/are selected and collected in the selecting step described below on the basis of the results of the detecting step, thus making it possible to reduce the labor of subculturing all the cells separately during the detecting step. In addition, this makes it possible to decrease the number of vessels to be used in cell culture and reduce the labor for cell culture, compared with inoculating cells at a lower concentration followed by subjecting all the cells to the detecting step.

As used herein, a “nucleic acid” refers to an organic polymer compound having a nitrogen-containing base derived from a purine or pyrimidine, a sugar, and a phosphoric acid as a constituent unit, and also encompasses an analog of such a nucleic acid, and the like. The nucleic acid may be, for example, DNA, RNA, cDNA, or the like. The nucleic acid may be, for example, a genomic DNA of a biological species from which the above-mentioned cell is derived. The nucleic acid may be labeled so as to be detected.

The “one or more” in a sequence of one or more base(s) is not limited to any range, but for example may be 1 base or 2 bases or more, 3 bases or more, or 5 bases or more, and may be 50000 bases or less, 100 bases or less, 50 bases or less, or 10 bases or less. For example, a sequence of one or more base(s) may contain or consist of 2 bases to 50000 bases, 2 bases to 50 bases, or, for example, 3 bases to 10 bases. A sequence of one or more base(s) may be a single-nucleotide polymorphism (SNP), a single-nucleotide variant (SNV), an insertion & deletion (Indel), or a structural variant (SV).

As used herein, a “single-nucleotide polymorphism (SNP)” means a variation between the genomes of the same species of individuals, and refers to such a variation the frequency of which is usually found to be approximately 1% or more in the population. The SNP can be an addition, deletion, or substitution of a base, and encompasses not only a mutation of 1 base but also a mutation of approximately 2 bases to 10 bases. In general, the SNP relatively frequently occurs in the genome, and contributes to genetic diversity.

As used herein, a “single-nucleotide variant (SNV)” means a variation between the genomes of the same species of individuals, and refers to a substitution of one base.

As used herein, an “insertion or deletion (Indel)” means a variation between the genomes of the same species of individuals, and refers to a short insertion or deletion of one base or more and less than 50 bases.

As used herein, a “structural variant (SV)” means a variation between the genomes of the same species of individuals, and refers to an insertion, deletion, duplication, translocation, inversion, or tandem repeat of 50 bases or more.

In one embodiment, SNP, SNV, Indel, and SV may be a mutation which causes a disease.

A nucleic acid detection method, for example, a SNP detection method is well-known to a person skilled in the art, and any such method may be used. Examples of a nucleic acid detection method include a single-base mismatch detection PCR, enzyme mismatch cleavage method (EMC), restriction fragment length polymorphism method (RFLP), TaqMan PCR method, indel detection by amplicon analysis method (IDAA), mass spectrometry method, direct sequencing, allele-specific oligonucleotide dot blotting method, single-base primer extension method, invader method, quantitative real-time PCR detection method, and the like. Typical nucleic acid detection methods out of the above-mentioned methods will be illustratively described below.

Single-Base Mismatch Detection PCR

Single-base mismatch detection PCR means PCR that is capable of detecting a single-base mismatch. Single-base mismatch detection PCR utilizes the fact that, in a case where a specific polymerase is used, and if a single base at the 3′ end of a primer does not completely match (if a mismatch exists), the amplification efficiency markedly decreases. Single-base mismatch detection PCR is performed using a sequence-specific primer with a corresponding base designed at the 3′ end. Single-base mismatch can be performed, for example, using HiDi DNA polymerase (Drum, M. et al., 2014, PLoS One, 9, e96640). When detection of a polymorphism or the like is performed, single-base mismatch detection PCR is also referred to as an “amplification refractory mutation system (ARMS)”, “allele-specific amplification (ASA)”, or “allele-specific PCR”.

Enzyme Mismatch Cleavage (EMC)

In an enzyme mismatch cleavage method, a heteroduplex is first formed by hybridizing a nucleic acid containing or not containing a mismatch when hybridized with a nucleic acid to be detected, followed by treatment with an enzyme cleaving a single-stranded region of a duplex that occurs if there is a mismatch. A mismatch region can be digested enzymically, for example, by treating with RNase for an RNA/DNA duplex, or treating with Si nuclease, CEL I endonuclease, T7 endonuclease I (T7E1), T7 endonuclease IV (T7E4), endonuclease V, Surveyor nuclease, or the like for a DNA/DNA hybrid. After the mismatch region is digested, the resulting product is separated according to size in a denaturing polyacrylamide gel so that a specific base sequence such as a SNP can be detected. For details, see, for example, Cotton, et al., 1988. Proc. Nat. Acad. Sci. USA 85: 4397, and the like.

Restriction Fragment Length Polymorphism (RFLP)

In cases where a nucleic acid sequence comprising a base sequence to be detected comprises a restriction enzyme recognition site, a restriction fragment length polymorphism analysis method (RFLP: Botstein, D. R., et al., Am. J. Hum. Gen., 32, 314-331 (1980)) can be used for the detection. In an RFLP method, a DNA fragment in a region comprising a base sequence to be detected in a nucleic acid is first amplified by a PCR method or the like to obtain a sample. Then, this sample is digested using a particular restriction enzyme, and the cleavage of the DNA (the occurrence or nonoccurrence of cleavage, the base length of a cleaved fragment, and the like) is examined by a conventional method to detect a particular base sequence such as a SNP.

TaqManPCR Method

TaqManPCR is a method that utilizes PCR using a fluorescence-labeled allele-specific oligonucleotide (TaqMan probe) and a TaqDNA polymerase (for example, Genet. Anal., 14, 143-149 (1999)). A TaqMan probe is an oligonucleotide of approximately 13 to 20 bases comprising a polymorphic site, and has the 5′ end labeled with a fluorescence reporter dye and the 3′ end labeled with a quencher. Using this allele-specific probe makes it possible to detect a particular base sequence such as a SNP.

Indel Detection by Amplicon Analysis (IDAA)

In IDAA, a target site is amplified using three primers: target-specific primers (F/R) flanking the target site and a 5′ FAM-labeled primer (FamF) specific for a 5′ overhang sequence attached to the F primer. As a result of the amplification, an FAM-labeled amplification product is obtained. Subsequently, detecting a fluorescence-labeled amplification product containing an indel by fragment analysis makes it possible to detect the indel. For details, see, for example, Yang Z. et al., 2015, Nucleic Acids Res., 43, e59; 1-8.

Mass Spectrometry

Mass spectrometry is a method that detects a difference of mass due to a difference of the base sequence. Specifically, PCR amplification of a region containing a base sequence to be detected is followed by hybridization of a primer for extension immediately before the position of a particular base sequence such as a SNP to perform an extension reaction. The extension reaction results in generating a fragment whose 3′ end differs depending on a SNP or the like. This generated product is purified and analyzed by mass spectrometer such as MALDI-TOF or the like, thus making it possible to analyze the correspondence between the mass number and the genotype, and detect a particular base sequence such as a SNP (see, for example, Pusch, W. et al., 2002, Pharmacogenomics, 3 (4): 537-48).

Direct Sequencing

Direct sequencing is a method in which PCR amplification of a DNA fragment comprising a particular base sequence is followed by sequencing of the nucleotide sequence of the amplified DNA directly by a dideoxy method or the like (Biotechniques, 11, 246-249 (1991)). A PCR primer used in this method is an oligonucleotide of approximately 15 to 30 bases, and a DNA fragment of approximately 50 bp to 2000 bp usually comprising a polymorphic site is amplified. In addition, a sequence primer to be used is an oligonucleotide of approximately 15 to 30 bases corresponding to the position approximately 50 to 300 nucleotides toward the 5′ end from the site to be sequenced.

Protein Detection Method

It is also possible to indirectly detect a base sequence by detecting a protein comprising an amino acid(s) specified by a sequence of one or more base(s) in a nucleic acid. Examples of protein detection methods include Western blotting. For example, in cases where the amino acid sequence specified by a base sequence differs by a SNP, for example, performing Western blotting using an antibody capable of identifying a single amino acid substitution makes it possible to detect the SNP indirectly.

In a method according to the present embodiment, the plurality of colonies is cultured in a culture vessel during the detecting step. The culture conditions during the detecting step are the same as described for the colony-forming step described above except for the culture period. The culture period during the detecting step is not limited provided that the period does not allow individual colonies in the culture vessel to be contaminated through their growth. The period may be, for example, 1 hour or more, 2 hours or more, 4 hours or more, 8 hours or more, 16 hours or more, 1 day or more, or 2 days or more, and may be 6 days or less, 5 days or less, 4 days or less, or 3 days or less. For animal cells, the period may be 4 days or less or 3 days or less. The culture period may be, for example, 4 hours to 6 days, 8 hours to 5 days, 1 day to 4 days, or 2 days to 3 days.

In one embodiment, the detecting step comprises a step in which a colony subjected to a nucleic acid or protein detection method (first screening) is further subjected to another gene or protein detection method (secondary screening). A combination of gene or protein detection methods used for the first screening and the secondary screening may be a combination of the above-mentioned nucleic acid or protein detection methods, or may be a combination of the above-mentioned nucleic acid or protein detection method and another nucleic acid or protein detection method. For example, in the first screening, an examination may be performed by a relatively simple method such as single-base mismatch detection PCR, EMC, RFLP, TaqMan PCR, IDAA, mass spectrometry, allele-specific oligonucleotide dot blotting, single-base primer extension, an invader method, or quantitative real-time PCR detection, and in the secondary screening, an examination may be performed by a more accurate method such as direct sequencing.

c) Selecting Step

In the selecting step, a colony from which the above-mentioned sequence of one or more base(s) has been detected is selected and collected out of the part of the colonies on the basis of the results of the above-mentioned detection method in the detecting step.

The method of selecting a colony from which the above-mentioned sequence of one or more base(s) is detected on the basis of the results of the above-mentioned detection method is not limited. For example, in cases where a vessel having an identifier capable of identifying the position of each colony in the vessel is used in the colony-forming step, the colony can be identified and selected on the basis of this identifier. Alternatively, each colony can also be identified by acquiring image information of a culture vessel containing a plurality of colonies formed therein and by using the visual information such as the coordinates, positional relationship, and size of each colony, and, if necessary, by analyzing the information with a computer.

The method of collecting the selected colony is not limited. For example, in cases where the collection is performed manually, the whole or part of the colony can be collected by peeling the part of the colony of interest, for example, by pipetting with a Pipetman tip, a Pasteur pipette, or the like under a microscope. Alternatively, the cells may be physically dissociated using a cell scraper or the like, and collected. In cases where an automatic or semi-automatic device is used, the collection can be performed using a robot arm in the same manner as performed manually.

Other Steps

A method of producing a culture cell according to the present aspect may comprise other steps in addition to the above-mentioned colony-forming step, detecting step, and selecting step. Examples of other steps include, but are not limited to, one or more of a genome-editing step, selection step, step of culturing a cell selected in the selecting step, and a step of examining the clonality of a cell, as described below. A method of producing a culture cell described herein may include or consist of the above-mentioned steps.

In one embodiment, a method of producing a culture cell according to the present invention comprises a step of performing genome editing on the above-mentioned animal cell or plant cell (hereinafter also referred to as a “genome-editing step”), for example, before the colony-forming step. As an example, in the genome-editing step, a mutant base sequence such as a SNP or SNV in the genomic DNA can be substituted with a wild-type base sequence, or the wild-type base sequence can be substituted with a mutant base sequence such as a SNP or SNV. Alternatively, a particular gene, a group of gene, or a group of natural or artificial base sequence associated therewith may be, for example, knocked in or knocked out, for example, at a specific site on a genome. Additionally, in the genome-editing step, a plurality of gene loci may be deleted on a large scale, all or part of a particular gene or a group of gene may be translocated into another chromosome, or a particular gene may be additionally duplicated in another chromosome, or may be duplicated in tandem at a flanking position in the same chromosome.

As used herein, a “wild-type” allele refers to an allele that naturally occurs the most in an allele population of base sequences in the same species, and has an original function if a protein or a non-coding RNA that is coded by the allele has a function. Herein, examples of types of mutations include a substitution, insertion, deletion, and structural variation (duplication, translocation, inversion, tandem repeat, and copy number variation).

In one embodiment, a mutant base sequence such as a SNP, SNV, Indel, or SV can be a cause of a disease. The type of disease is not limited provided that the disease can be caused by the mutant base sequence. Examples of disease include multiple endocrine neoplasia type 2B (multiple endocrine neoplasia type 2A (MEN2B)), multiple endocrine neoplasia type 2A (MEN2A), multiple endocrine neoplasia type 1 (MEN1), dystrophic epidermolysis bullosa (DEB), hereditary breast and/or ovarian cancer syndrome (HBOC), Li-Fraumeni syndrome (LFS), Cowden syndrome, Lynch syndrome, familial adenomatous polyposis (FAP), hyperparathyroidism-jaw tumor syndrome (HPT-JT), amyotrophic lateral sclerosis (ALS), myotonic dystrophy 1 ((DM1), familial Parkinson's disease, hereditary Alzheimer's disease, Marfan's syndrome, metatrophic dysplasia, fibrodysplasia ossificans progressiva, neonatal-onset multisystem inflammatory disease (NOMID), FGFR3 chondrodysplasia, type II collagenopathy, von-Hippel-Lindau disease (VHLD), Citrin deficiency, transthyretin-type familial amyloid polyneuropathy, Niemann-Pick type C (NPC), Charcot-Marie Tooth disease, Tay-Sachs disease, Williams syndrome, Duchenne muscular dystrophy, Smith-Magenis syndrome, Camey complex, Alzheimer's disease due to APP mutation, Potocki-Lupski syndrome, Prader-Willi syndrome, Angelman syndrome, Down's syndrome, XX male syndrome (SRY), schizophrenia (chr 11), Burkitt's lymphoma, Hemophilia A, Hunter syndrome, Emery-Dreifuss muscular dystrophy, fragile X syndrome due to FMR1 mutation, Huntington's disease, spinocerebellar ataxia, and the like.

As used herein, “genome editing” refers to a technology for specifically cleaving and editing a target site on a genome. In genome editing, a site-specific nuclease (SSN) capable of sequence-specific cleavage (herein, also referred to as a “genome editing protein”), for example, TALEN (transcription activator-like effector nuclease), CRISPR/Cas9 (clustered regularly interspaced short palindromic repeats CRISPR)/CRISPR-associated protein 9), CRISPR/Cpf1 (CRISPR from Prevotella and Francisella 1), ZFN (zinc finger nuclease), a modified protein thereof, or the like is used. Examples of SSN include other bacteria-derived analogs (SaCas9, ScCas9, FnCpf1, and the like) in the CRISPR/Cas system, other Cas protein groups (Cas12a, Cas12b, C2c1, C2c2, C2c3, and the like), and a group of modified proteins thereof. A site-specific nuclease (SSN) having an accurate target-recognition ability and capable of identifying a single-base substitution (SSN) is preferably used. Examples of such nucleases include the following: TALEN and modified protein thereof (for example, Platinum TALEN); Streptococcus pyogenes Cas9 (SpCas9) and modified protein thereof (for example, high-fidelity modified proteins such as eSpCas9-1.0/-1.1, SpCas9-HF1/HF2/HF3/HF4, HypaCas9, and xCas9), and PAM modified protein of SpCas9 (SpCas9 (VQR), SpCas9 (EQR), SpCas9 (VRER), SpCas9 (D1135E), SpCas9 (QQR1), and the like); Staphylococcus aureus Cas9 (SaCas9) and modified protein thereof (for example, SaCas9HF andSaCas9 (KKH)); Streptococcus canis Cas9 (ScCas9) and modified protein thereof (for example, ScCas9HF); Acidaminococcus sp. Cpf1 (AsCpf1) and modified protein thereof (for example, AsCpf1 (RR) and AsCpf1 (RVR)); Lachnospiraceae bacterium ND2006 Cpf1 (LbCpf1) and modified protein thereof (for example, LbCpf1 (RR) and LbCpf1 (RVR)); Francisella novicida Cpf1 (FnCpf1) and modified protein thereof (for example, FnCpf1 (RR) and FnCpf1 (RVR)); Moraxella bovoculi 237 (Mb) Cpf1 and modified protein thereof (for example, MbCpf1 (RR) and MbCpf1 (RVR)); and the like.

In genome editing, a DNA cleaved by the above-mentioned nuclease or the like is repaired through homology directed repair or non-homologous end-joining, during which a gene of interest can be modified. Using a nuclease that has an accurate target-recognition ability in genome editing, for example, AsCpf1 makes it possible to enhance the accuracy with which a cell of interest is obtained in a subsequent screening or the like.

In cases where a genome editing protein is CRISPR/Cas9, CRISPR/Cpf1, another Cas protein group, or the like, genome editing (cleavage) necessitates simultaneously introducing crRNA (or guide RNA) in addition to a genome editing protein.

In cases where a genome editing protein is CRISPR/Cas9, CRISPR/Cpf1, another Cas protein group, or the like, genome editing (intended substitution, insertion, deletion, or the like) necessitates simultaneously introducing a template DNA (ssODN, dsDNA, or the like) in addition to a genome editing protein and crRNA (or guide RNA).

Herein, the genome-editing step includes base substitution using a cytosine base editor (CBE) or adenine base editor (ABE) in addition to a genome editing method using a general SSN and a template DNA. A CBE is an artificial enzyme based on a CRISPR/Cas protein that enables single-base transition from a G-C base pair to a T-A base pair. Examples of proteins that can be used include CBE and modified proteins and analogues thereof (for example, BE1, BE2, BE3, HF-BE3, BE4, BE4max, BE4-gam, YE1-BE3, EE-BE3, YE2-BE3, YEE-BE3, VQR-BE3, VRER-BE3, SaBE3, SaBE4, SaBE4-Gam, Sa(KKH)-BE3, Cas12a-BE, Target-AID, Target-AID-NG, xBE3, eA3A-BE3, A3A-BE3, BE-PLUS, TAM, and CRISPR-X) and the like. On the other hand, an ABE is an artificial enzyme based on a CRISPR/Cas protein that enables single-nucleotide transition from an A-T base pair to a G-C base pair. Examples of proteins that can be used include ABE and modifications and analogues thereof (for example, TAM, CRISPR-X, ABE7.9, ABE7.10, ABE.7.10*, xABE, ABESa, VQR-ABE, VRER-ABE, and Sa(KKH)-ABE) and the like.

In addition to a genome editing protein (and optionally the above-mentioned crRNA), for example, a selection marker such as antibiotic resistance for selecting a genome-edited cell may be introduced into a cell.

A genome editing protein (and optionally a selection marker) may be introduced into a cell in the form of a vector comprising a nucleic acid that encodes these proteins and markers. In addition to the nucleic acid that encodes a genome editing protein, a nucleic acid and/or crRNA that encodes the selection marker may optionally be contained in the same vector, or may be contained in a plurality of different vectors. In addition, the vector may be introduced into a cell together with e.g., a single-stranded DNA or double-stranded DNA, for example, a single-stranded oligodeoxynucleotide, as a modification template that can be incorporated into DNA after cleavage. The vector can be introduced into a cell by a known method. Examples of such an introducing method include an electroporation method, sonoporation method, particle gun method, lipofection method, PEG-calcium phosphate method, polyethyleneimine (PEI)-mediated transfection method, and microinjection method, viral vector method, and the like.

In one embodiment, a “marker base” in addition to a base intended for substitution is introduced in the genome-editing step. A “marker base” is a base that is added in addition to a base to be edited in order to simplify detection in the subsequent detecting step. In cases where a marker base is used, detecting the presence of the marker base makes it possible to indirectly detect a clone that has undergone editing of interest. A marker base to be generally used is a base that produces a silent mutation having no influence on the amino acid sequence, but even a silent mutation can have an influence on the expression efficiency of a protein. Thus, to decrease the risk of side effect, it is preferable to introduce no marker base.

In one embodiment, the genome-editing step involves detecting the absence of a nucleotide existing before substitution by genome editing in the detecting step without introducing a marker base (negative screening). Previously, negative screening using no marker base was generally considered to be difficult. Contrary to such common technical knowledge, the present inventors have found that it is possible to select a genome-edited cell with sufficient efficiency without introducing a marker base though negative screening which was previously considered to be difficult. A method according to the present embodiment can achieve the effect of decreasing the risk that a marker base can influence the expression efficiency of a protein, thus reducing the disadvantage of side effect. Furthermore, the method can be applied to the introduction and/or repair of a mutation in an untranslated region involved in transcriptional regulation, splicing control, and the like.

In one embodiment, a method according to the present invention may comprise a selection step before the colony-forming step and after the genome-editing step. The selection step can be performed, for example, by introducing a selection marker such as antibiotic resistance into a cell at the same time with the genome-editing step, and culturing the cell in the presence of this antibiotic resistance and the like. Examples of antibiotics include puromycin, neomycin, blasticidin, hygromyocin, and zeocin, and the kind and concentration of such antibiotics can be determined as appropriate by a person skilled in the art. In addition, the culture condition in the selection step can be the same as described for the above-mentioned colony-forming step except for the culture period. The culture period can be, for example, 1 day to 7 days, 2 days to 5 days, or 2 days to 3 days.

In one embodiment, a method according to the present invention comprises a step of culturing a cell selected and collected in the selecting step (hereinafter referred to as a “culturing step”), after the selecting step. The culture condition in the culturing step can be the same as described for the above-mentioned colony-forming step except for the culture period. The culture period can be, for example, 1 day to 14 days, 2 days to 7 days, or 3 days to 4 days. In the culturing step, it is also possible to perform a subculturing step and thus perform the culture for a longer period than the above-mentioned period.

In one embodiment, a method according to the present invention comprises a step of examining the clonality of a cell. A method of examining the clonality is not limited, but may be performed by sequencing a region (e.g., approximately 50 bp, approximately 100 bp, or approximately 200 bp) containing a genome-edited sequence, e.g., through direct sequencing, and investigating the presence or absence of an indel. For example, it can be an indicator of the occurrence of clonal proliferation that there is variability of indel around a target site among the unintended gene-edited clones, and/or that no indel is generated in the intended gene-edited clones.

2. Method of Producing a Culture Cell without Using a Marker Base

In one aspect, the present invention relates to a method of producing a culture cell, comprising the steps of: (i) substituting a mutant base sequence with a wild-type base sequence or substituting a wild-type base sequence with a mutant base sequence in the genomic DNA in an animal cell or plant cell by genome editing without introducing a marker base (hereinafter also referred to as a “genome-editing step”); (ii) detecting the absence of a nucleotide existing before substitution by the genome editing in the genome-edited cell (hereinafter also referred to as a “detecting step”); and (iii) selecting and collecting a cell in which the absence of the nucleotide existing before substitution is detected (hereinafter also referred to as a “selecting step”).

The genome-editing step in the present embodiment is the same as the genome-editing step described in “1. Method of producing a culture cell” except that no marker base is introduced. In addition, the detecting step and the selecting step in the present embodiment are the same as the detecting step and the selecting step respectively described in “1. Method of producing a culture cell” except that it relates to genome editing, and, for example, the detection can be performed through examination by one or more methods selected from the group consisting of single-base mismatch detection PCR, EMC, RFLP, TaqMan PCR, IDAA, mass spectrometry, an allele-specific oligonucleotide dot blotting method, single-base primer extension method, invader method, quantitative real-time PCR detection method, and direct sequencing.

A method according to the present aspect may optionally comprise a selection step and/or a colony-forming step after the genome-editing step, and in addition, may comprise a step of culturing a cell selected in the selecting step and/or a step of examining the clonality of a cell after the selecting step. The selection step, colony-forming step, step of culturing a cell, and step of examining the clonality of a cell are the same as the respective steps described in “1. Method of producing a culture cell”.

3. Other Aspects

In one aspect, the methods described in “1. Method of producing a culture cell” and “2. Method of producing a culture cell without using a marker base” can be described as a method of selecting a cell (or colony) from which the above-mentioned sequence of one or more base(s) is detected, a method of selecting a genome-edited cell (or colony), or a method of producing such a cell.

In one aspect, the present invention relates to a cell obtained by the method described in “1. Method of producing a culture cell” or “2. Method of producing a culture cell without using a marker base,” for example, a genome-edited cell. In one aspect, the present invention relates to a pharmaceutical composition comprising the above-mentioned genome-edited cell, for example, a pharmaceutical composition for treating and/or preventing a disease, and use of the cell for treating and/or preventing a disease.

In one aspect, the present invention relates to a method of treating a disease, comprising the steps of: obtaining a cell genome-edited by a method according to the present invention; and treating and/or preventing a disease using the obtained cell.

Treatment and/or prevention of a disease in these aspects can be performed, for example, by growing a genome-edited cell, differentiating the cell if necessary, and administering or transplanting the cell into a subject. In this embodiment, a cell to be genome-edited can be a stem cell obtained from a subject, in order to reduce rejection in the subject.

In these aspects, a subject that can undergo treatment and/or prevention may be a human. In addition, the disease may be, but is not limited to, a disease that can be caused by a mutation such as a SNP, SNV, Indel, or SV, and may be e.g., a disease described herein, such as multiple endocrine neoplasia type 2B (MEN2B) or dystrophic epidermolysis bullosa (DEB).

### EXAMPLES

<Materials and Methods>

Experimental Flow

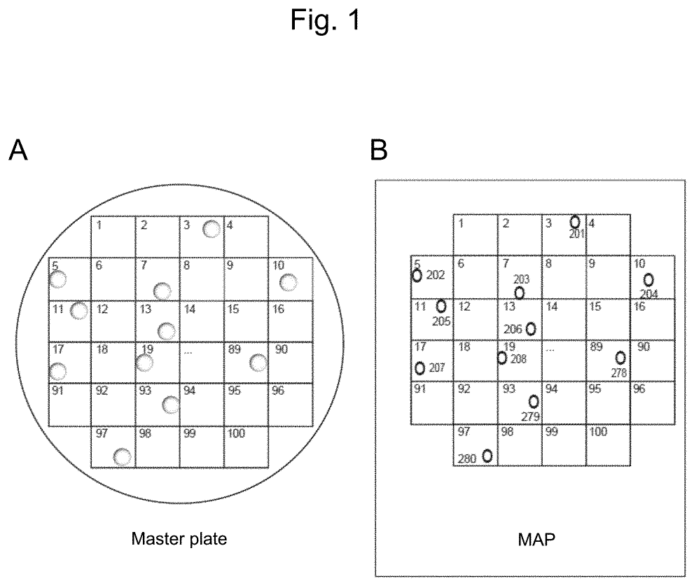

The experimental flow is as follows. To perform homology directed repair (HDR), an all-in-one vector (pY211-puro) that expresses a genome editing tool (ASCpf1-RR), crRNA, and a puromycin resistance gene, and an ssODN template were introduced into a human iPSC by electroporation. After selection by puromycin and collection, single cells were plated sparsely on a 100 mm plate having grid (master plate), and cultured for 7 to 8 days until colonies each of which was derived from a single cell were formed on the plate. FIG. 1 shows a schematic of a master plate used in Examples. The master plate has a plurality of sections divided by grid so that clones can be identified during the below-mentioned first or secondary screening and during subsequent collection (in the figure, the sites subjected to the screening are each marked with ∘; and Figure TA shows the master plate, and FIG. 1B is a map prepared therefrom). Subsequently, genomic DNA was extracted from a sample derived from a part of the colonies, and subjected to the first screening (to identify a colony that would serve as a candidate for single-base mismatch detection PCR). During the first screening, the master plate containing both the extracted colonies and the residual colonies were maintained as it was. In cases where positive screening was performed in the first screening, ssODN was used to introduce a single-base (single-nucleotide) marker (S) in addition to an intended substitution (M) (MS template), and the presence of a marker base was detected by single-base mismatch detection PCR. In cases where negative screening was performed, ssODN was used to introduce only an intended substitution (M) (M template), and the absence of the base existing before substitution was examined by single-base mismatch detection PCR. Then, the colonies that passed through the first screening was subjected to secondary screening by direct sequencing. In addition, the colonies which had passed the secondary screening were collected from the master plate using a pipette tip under a microscope, and the proliferated clones were tested by Sanger sequencing for the efficiency of the introduction of an intended mutation. The details of the experiment are described below.

Design and Construction of AsCpf1_RR and CRISPR RNA (crRNA)

To produce an all-in-one vector of an AsCpf1-RR mutant having a puromycin resistance gene, a pY211 mammalian expression vector (Gao, L. et al., 2017, Nat. Biotechnol., 35, 789-792) (Addgene plasmid number 89352, obtained from Dr. Feng Zhang) containing an AsCpf1-RR and crRNA backbone was used. Here, the 3×HA tag was substituted with a 3×HA tag, a T2A peptide cDNA, and an SGFP2 cDNA. Briefly, 3×HA and T2A fragments were constructed by annealing of ssODN, and SGFP2 cDNA was amplified from pSGFP2-C1 (Addgene plasmid number 22881, obtained from Dr. Dorus Gadella). The pY211 vector was cleaved with BamHI/EcoRI. Finally, all the fragments were fused using an In-Fusion HD Cloning kit (Clontech/Takara Bio Inc.). The resulting plasmid was named pY211-T2G. Then, a cDNA corresponding to the puromycin resistance gene was amplified from a pSIH-H1-Puro vector (Addgene plasmid number 26597, obtained from Dr. Frank Sinicrope) was fused with the pY211-T2G vector at the SpeI/EcoRI site. The final all-in-one vector was named pY211-puro.

The crRNA was manually designed as described earlier (Gao, L. et al., as above). A crRNA guide sequence template targeting RET exon 16 or COL7A1 exon 78 was cloned, as described earlier, into a pY211-puro vector digested with BbsI (Gao, L. et al., as above).

Design of ssODN Repair Template

An ssODN template of 99 base in length for repair and modification (PAGE-purified, from Sigma-Aldrich Co. LLC) had the 5′ predicted cleavage site of CRISPR-AsCpf1 at the center and contained 5′ flanking and 3′ flanking regions of 49 base in length. This template contained a pathogenic SNP/SNV or a wild-type nucleotide corresponding thereto, and optionally contained a silent mutation to be used as a marker base. The silent mutation was selected on the basis of the codon usage database (https://www.kazusa.or.jp/codon/).

Construction of iPS Cells

iPSCs were produced from human T cells using CytoTune-iPS 2.0 Sendai Reprogramming Kit (Thermo Fisher Scientific Inc., U.S.A.) in accordance with the protocol of the manufacturer. Briefly, the human T cells were isolated from a peripheral blood sample, and inoculated at a density of 1.5×10⁶ cells/well in a 6-well plate coated with an anti-CD3 antibody (eBioscience). After one day, 3×10⁵ cells were infected at a multiplicity of infection (MOI) of 10 with a recombinant Sendai virus (SeV) having a reprogramming factor. After culturing for 2 days, the infected cells were collected and inoculated again at 2×10⁴ cells per a 100 mm dish into mitomycin C (MMC)-treated mouse embryonic fibroblasts (which served as feeder cells). After 20 to 23 days from the infection, colonies were collected, and cultured again on a human iPSC culture medium (the details are as described below). To remove SeV, the iPSCs were cultured at 38° C. for three days, and passaged once.

To examine the quality of the newly established iPSCs (FB4-14 and B117-3 cells), five pluripotency genes (Nanog, Gdf3, Rex1, Sa114, and Dnmt3B) and four Yamanaka factors (Oct-3/4, Sox2, Klf4, and c-Myc) were examined by RT-PCR using a primer set designed before.

Cell

In gene-editing (GE) experiments 1-4 (GE1-4), an FB4-14 cell having an autosomal dominant mutation at a disease gene locus (MEN2B-specific iPSC) was used. This gene locus has a single allele point mutation (from T to C) at the RET gene in the codon 918 of the exon 16, which leads to a Met918Thr substitution. In GE5, a B117-3 cell having an autosomal recessive complex mutation at a disease gene locus (DEB-specific iPSC) was used. This gene locus has a single allele point mutation (from G to T) that leads to a nonsense mutation (G2138X) in the COL7A1 gene at the codon 2138 of the single exon 78, and in addition, has a single allele indel (n. 3591 del. 13, ins. GG) that results in a frameshift.

iPSC Culture

Cells were maintained in a cell culture plate coated with 0.1% gelatin inoculated with MMC-treated MEFs, and allowed to proliferate on an iPSC culture medium under 5% CO₂ at 37° C. This culture medium consists of DMEM/F12 (Sigma-Aldrich Co. LLC) supplemented with 20% KNOCKOUT™ serum substitute (KSR, from Invitrogen), 2 mM L-glutamine (Life technologies), 0.1 mM non-essential amino acid (NEAA, from Sigma-Aldrich Co. LLC), 0.1 mM 2-mercaptoethanol (Sigma-Aldrich Co. LLC), 0.5% penicillin and streptomycin (Nacalai Tesque, Inc.) and 5 ng/ml basic fibroblast growth factor (bFGF, from Fujifilm Wako Pure Chemical Corporation). Before transfection, the iPSCs were transferred to a cell culture plate coated with Matrigel-GFR (Corning), and cultured for two days on an iPSC culture medium (MEF-CM) prepared with MEF containing 10 μM ROCK inhibitor. Lastly, the cells were allowed to proliferate under 5% CO₂ at 37° C. on an mTeSR1 culture medium (Stemcell Technologies Inc.) in a cell culture plate coated with Matrigel-GFR.

Transfection

A pY211-puro vector (an all-in-one mammalian expression vector containing AsCpf1-RR cDNA, CRISPR RNA, and a puromycin resistance gene) and ssODN were electroporated into the iPSC. Briefly, 1×10⁶ cells were re-suspended in 100 μl of OptiMEM containing 10 μg of pY211-puro and 15 μg of ssODN (99 nt, PAGE-purified; from Sigma-Aldrich Co. LLC). Subsequently, Super Electroporator NEPA21 Type 2 (Nepa Gene Co., Ltd., Japan) was used to electroporate the cells in a 2-mm gap cuvette (transfer pulse, 20 V; pulse length, 50 ms; and pulse number, 5). After the electroporation, the cells were transferred to a Matrigel-GFR-coated 24-well plate, allowed to grow on an mTeSR1 culture medium containing CloneR (Stemcell Technologies Inc.) for 16 hours, treated on an mTeSR1 culture medium containing CloneR and puromycin (0.5 μg/ml) for 48 hours, and allowed to grow on an mTeSR1 culture medium containing CloneR for one to two days. Then, the cells were made into single with TrypLE (Thermo Fisher Scientific Inc., U.S.A.), inoculated at a low density (500 to 1000 cells per a 100-mm plate) into an mTeSR1 culture medium containing CloneR, and cloned.

Genotype Determination of Clones Derived from Single Cells by Single-Base Mismatch Detection PCR and Sequencing

To facilitate determination of the genotype of a clone derived from a single cell, an ssODN template was designed for HDR in positive screening, and the ssODN template was constructed to produce a silent single-base substitution (SNS) independent of a pathogenic SNP and mutation which is applicable to single-base mismatch detection PCR. The iPSC clones derived from the genome-edited single cells were allowed to grow on an mTeSR1 culture medium containing CloneR for four days, and on an mTeSR1 culture medium for three days, in a master plate (Matrigel-GFR-coated 100-mm plate having 100 square grids (PetriSticker, from Diversified Biotech Inc.)). Approximately 25 to 33% of the colonies grown were manually collected using a clean 10-μL pipette tip under a binocular stereoscopic microscope, and directly transferred to a PCR tube containing 10 μl of lysis buffer. The master plate containing the residual colonies were maintained as they were under the same conditions for two to three days.

The genomic DNA was extracted by a proteinase K method. Briefly, the cell sample was re-suspended in 10 μL of lysis buffer, and incubated at 55° C. for 12 hours. Proteinase K was inactivated by heat treatment at 85° C. for 45 minutes. Using single-base mismatch detection PCR to identify a clone of interest having an occurrence of HDR, the genome region around the target gene locus was amplified using an HiDi DNA polymerase (Drum, M. et al., 2014, PLoS One, 9, e96640) (myPOLS Biotec GmbH, Germany) and a corresponding allele-specific primer pair. The amplification product was analyzed by 2% agarose gel electrophoresis. The marker base introduced by HDR and the zygosity of the pathogenic mutation in the clone were confirmed by Sanger sequencing. That is, 450 to 500 bp around the gene-edited gene locus were amplified by PCR using a specific primer pair and a Tks Gflex DNA polymerase (Takara Bio Inc., Japan). Sequencing was performed using BigDye Terminator v3.1 Cycle Sequencing Kit (Thermo Fisher Scientific Inc., U.S.A.).

To examine the clonality of the gene-edited clones, the sequence of the unintended gene-edited clone that passed the first screening was analyzed. The unintended gene-edited clone contains several indels generated by NHEJ after the gene-editing. The composition of the unintended gene-edited clone directly indicates the clonality of unintended gene-edited clone. In addition, this indirectly indicates the clonality of the intended gene-edited clone. The sequence read of the unintended gene-edited clone was manually separated into single reads of normal alleles and alleles containing an indel. For the distribution of indel, the read containing an indel was analyzed by manual alignment with the reference sequence.

Off-Target Analysis

The gene-edited disease-specific iPSC was tested for the off-target event predicted for each guide RNA, using CHOPCHOP v2, which is a web-based CRISPR design tool (http://chopchop.cbu.uib.no) prepared by the laboratory of Eivind Valen. For each crRNA, seven top off-target sites predicted by the above-mentioned design tool were used. The genomic DNA region around each off-target site was amplified by PCR, and compared with RefSeq in the latest Human Dec. 2013 (GRCh38/hg38) assembly on the UCSC genome browser (http://genome.ucsc.edu/).

<Results>

Allele-Specific Single-Nucleotide Substitution Using a Marker Base

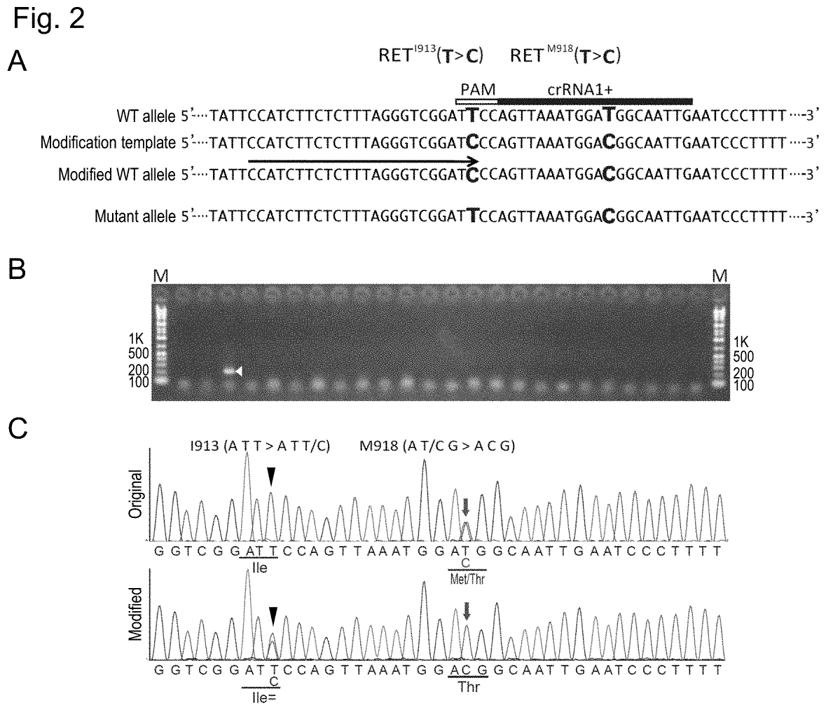

In this Example, a wild-type nucleotide at the RET_M918 site in a wild-type allele was substituted with a mutation nucleotide in FB4-14 MEN2B-iPSC to investigate the allele-specific single-nucleotide substitution activity in a human iPSC (FIG. 2A). First, AsCpf1_RR, crRNA1+, and furthermore, an ssODN modification template (ssODN_RET_M918T_1913_silentC (Mut)) having both a mutation base at M918 and a marker base at 1913 (the second line in FIG. 2A) were electroporated together into an FB4-14 cell (the first line in FIG. 2A). Out of 384 clones, 12 clones were found to be positive by single-base mismatch detection PCR analysis (FIGS. 2, A and B, and Table 1, GE1). The direct sequencing of the target sequence has revealed that 7 out of 12 clones were intended gene-edited clones having an introduction of a wild-type allele-specific mutation nucleotide at a target site (a substitution of T with C, leading to a substitution of Met with Thr at Met918; the arrows in FIG. 2C) and a marker base (a substitution of T with C, leading to a silent mutation at Ile913; the arrowheads in FIG. 2C). The HDR efficiency was 1.8% (Table 1). Subsequently, the web tool CHOPCHOP v2 was used to investigate the off-target target. As a result, no indel was detected at two predicted off-target sites (Table 2). Furthermore, that the sequence around the target site was different between the unintended gene-edited clones showed that most of the intended gene-edited clones underwent clonal proliferation (data not shown). These results showed that a single wild-type nucleotide can be substituted with a mutation nucleotide in an allele-specific manner without causing an off-target effect, as shown for a wild-type allele in MEN2B-iPSC.

Allele-Specific Single-Nucleotide Repair of Pathogenic Mutation Using a Marker Base

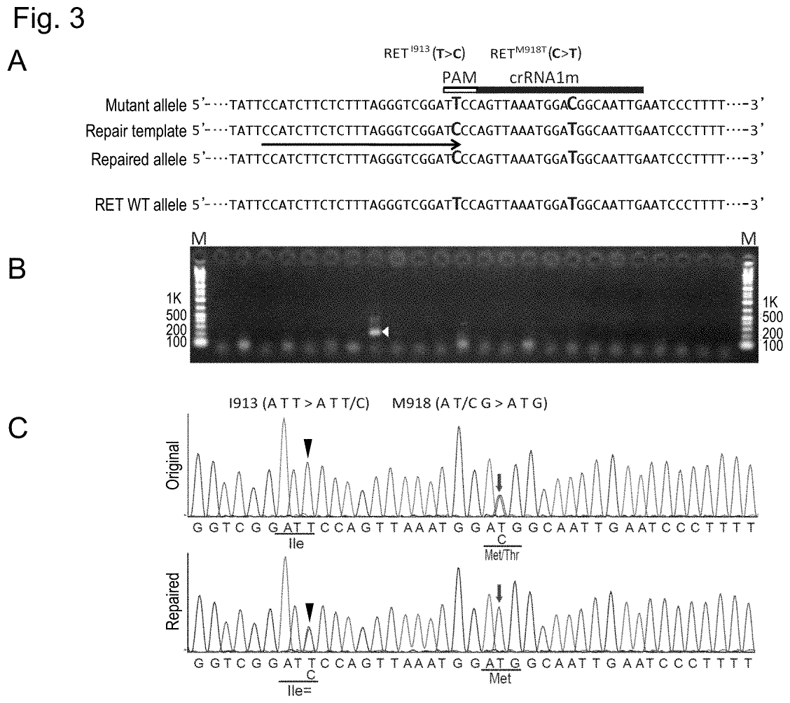

DSB/HDR was performed on FB4-14 cells using AsCpf1_RR, crRNAlm, and furthermore, an ssODN repair template containing a repair nucleotide at Met918 and a marker base at Ile913 (ssODN_RET_M918_I913_silentC (WT)) (FIG. 3A, second line). The resulting cells were subjected to single-base mismatch detection PCR, and 17 of 344 clones were found to be positive clones (FIG. 3B; and Table 1, GE2). In addition, direct sequencing has confirmed that 11 of these 17 clones were intended gene-edited clones. That is, these clones had a substitution of C with T from a mutant nucleotide to a wild-type nucleotide at a target site (leading to an amino acid substitution of Thr with Met at Met918) (FIG. 3C, arrows), and had a marker base introduced (leading to a silent mutation at Ile913) (FIG. 3C, arrowheads).

The overall HDR efficiency was 3.2% (Table 1, GE2), and no off-target effect was observed (Table 2, GE2). In addition, analysis by target resequencing around a target site using a next-generation sequencer has revealed that unintended contamination of genomic DNA derived from a parent cell did not occur in the gene-edited clones (data not shown), and this result show that the intended gene-edited clone underwent clonal growth.

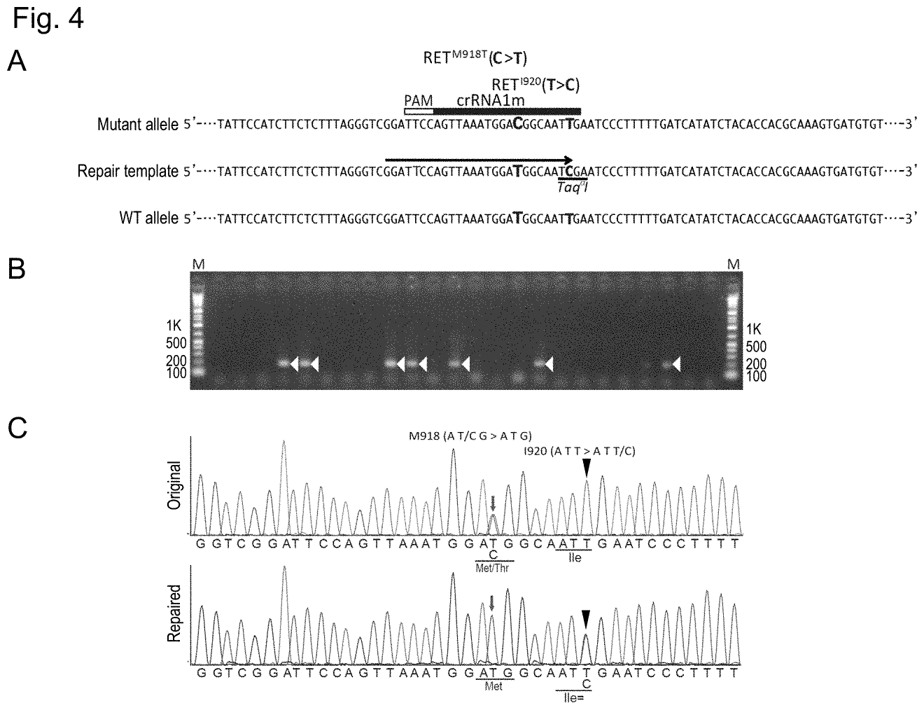

Then, DSB/HDR was performed using AsCpf1_RR, crRNA1m, and furthermore, an ssODN repair template containing a repair nucleotide at Met918 and a marker base at Ile920 (ssODN_RET_M918_1920_silentC (WT)) (FIGS. 4, A, B, and C; and Table 1, GE3). The use of the ssODN_RET_M918_1920_silentC (WT) also made it possible to carry out HDR in the same manner (Table 1), with no off-target effect observed (Table 2, GE3).

These results have revealed that a single-nucleotide substitution in a gene-edited clone can be efficiently detected by positive screening using single-nucleotide mismatch detection PCR.

Allele-Specific Single-Nucleotide Repair of a Pathogenic Mutation Using No Marker Base

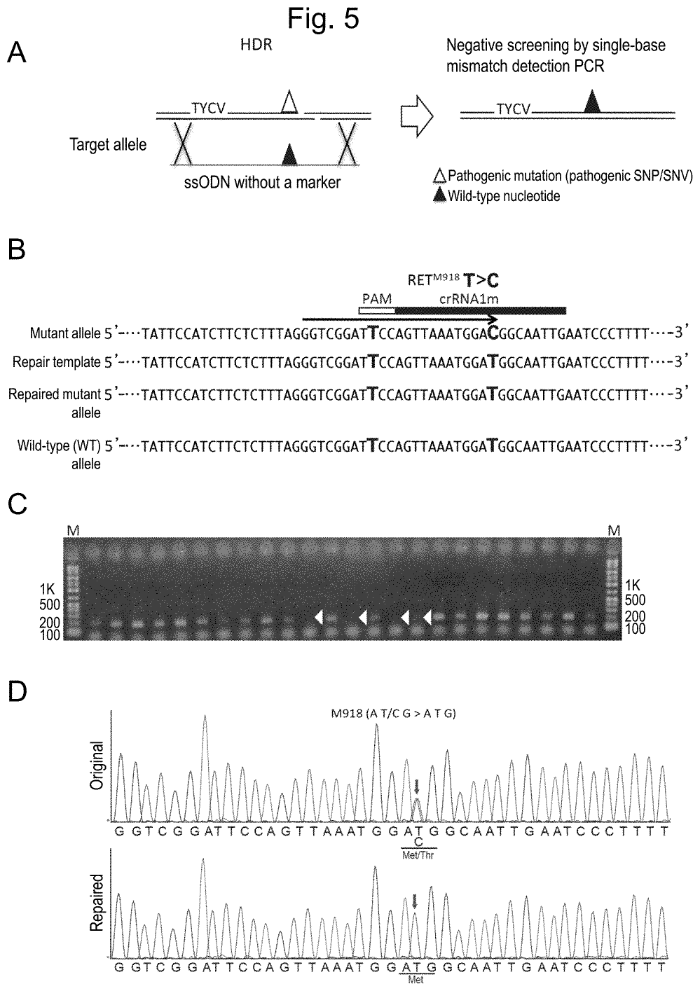

Subsequently, negative screening was performed on a pathogenic SNP at the RET_M918 site in a mutant allele of an FB4-14 cell without using a marker base (FIG. 5A). Single-base mismatch detection PCR was performed on a pathogenic SNP after the DSB/HDR in the FB4-14 cell using AsCpf1_RR, crRNA1m, and an ssODN repair template containing only a normal nucleotide at Met918 (ssODN_RET_M918 (WT)). In this experimental system, the SNP in the mutant allele disappears in the repaired clone, and thus, the repaired clone is detected as a negative clone by single-base mismatch detection PCR for a pathogenic SNP (FIGS. 5A and 5C). As a result, the single-base mismatch detection PCR for the pathogenic SNP identified 44 negative clones. Direct sequencing confirmed that 5 of these 44 clones were intended gene-edited clones, i.e., clones containing only a wild-type nucleotide at Met918 (FIGS. 5C and D; and Table 1, GE4). The overall HDR efficiency was 2% (Table 1), and in addition, no indel was detected at the predicted off-target site (Table 2, GE4). In addition, analysis by target resequencing around a target site using a next-generation sequencer has revealed that no unintended contamination of genomic DNA derived from a parent cell occurred in the gene-edited clones (background level, data not shown), showing that the intended gene-edited clone underwent clonal growth.

Subsequently, repair of a pathogenic SNP was performed in an iPSC derived from a patient with another disease, i.e., dystrophic epidermolysis bullosa (DEB). DEB is a hereditary disease characterized by serious relapsing skin ulceration and blister. DEB is caused by a genetic mutation in the human COL7A1 encoding type VII collagen which is an anchoring fibril binding the epithelium to the dermis.

iPSCs were produced from a patient with DEB, and the allele-specific nucleotide was substituted at the exon 78 target site of COL7A1^{G2138X/+; 3591 del.13, ins. GG/+}, which is an autosomal recessive complex mutation. As a result, HDR was also successfully performed and detected without introduction of a marker base for the above-described mutation (Table 1, GE5), and off-target effect was not observed (data not shown).

These results have revealed that a method according to the present invention makes it possible to perform a single-nucleotide repair of a pathogenic SNP in an intact manner without an off-target effect and without introducing a marker base serving as a landmark of genome editing.

TABLE 1
Gene-editing experiments

| | | | | Number of | Number of | |
|---|---|---|---|---|---|---|
| | | | Total | clones | clones | |
| | | | number of | that passed first | that passed | |
| | | | collected | screening (Single- | secondary | 2nd/ |
| Gene | | Genotype (phenotype) | clones | base mismatch | screening | TC |
| editing # | Cell | Original → Edited | (TC) | detection-PCR) | (sequencing) | (%) |
| GE1 | FB4-14 | RET^{M918T/+} → RET^{M918T/M918T; i913}_silentC/+ | 384 | 12* | 7 | 1.8 |
| | | (MEN2B^{a} → MEN2B homo with a | | | | |
| | | marker base) | | | | |
| GE2 | FB4-14 | RET^{M918T/+} → RET^{+/+; i913}_silentC/+ | 344 | 17* | 11 | 3.2 |
| | | (MEN2B^{a} → MEN2B revertant with a | | | | |
| | | marker base) | | | | |
| GE3 | FB4-14 | RET^{M918T/+} → RET^{+/+; i920}_silentC/+ | 336 | 30* | 19 | 5.7 |
| | | (MEN2B^{a} → MEN2B revertant with a | | | | |
| | | marker base) | | | | |
| GE4 | FB4-14 | RET^{M918T/+} → RET^{+/+} | 240 | 44** | 5 | 2.0 |
| | | (MEN2B^{a} → MEN2B intact revertant) | | | | |
| GE5 | B117-3 | COL7A1^{G2138X/+; 3591 del. 13, ins. GG/+} → | 80 | 18** | 6 | 7.5 |
| | | COL7A1^{+/+; 3591 del.} ^{13. ins. GG/+} (DEB^{b} → | | | | |
| | | DEB intact revertant) | | | | |

Electroporation of an RGEN expression vector together with an ssODN template was followed by single-base mismatch detection-PCR as the first screening, using crude DNA sample derived from colonies derived from single cells grown on the master plate.
In the positive screening, candidates were colonies that showed the fragment amplified by the single-base mismatch detection-PCR primer for detecting the marker (GE1 to GE3).
In the negative screening, candidates were colonies that exhibited no amplification (GE4 and GE5). The first screening was followed by directly reading the sequence around the target site of the DNA fragment amplified from each sample.
^{a}Multiple endocrine neoplasia type 2B
^{b}Dystrophic epidermolysis bullosa
^{c}Silent C refers to a silent mutation prepared by substitution with cytidine.
*Positive screening results
**Negative screening results

TABLE 2
Off-target effects in gene-editing experiments 1 to 4 (GE 1 to 4)

| | | | Number of | Sequence^{a} | SEQ ID | Indel ratio |
|---|---|---|---|---|---|---|
| Sample | | Genomic location | mismatch | (including mismatch) | NO: | (%)^{b} |
| Original | RET exon 16 | chr10: 43121953 | | TTCCAGTTAAATGGATGGCAATTG | 1 | |
| | target 1 | | | | | |
| GE1 | Off-target 1 | chr15: 91512242 | 3 | TTCCcGTTAAtTGGtTGGCAATTG | 2 | 0/7 (0%) |
| GE1 | Off-target 2 | chr4: 128631982 | 3 | TTCCAcTTAAATGcATGGCAtTTG | 3 | 0/7 (0%) |
| GE2 | Off-target 1 | chr15: 91512242 | 3 | TTCCcGTTAAtTGGtTGGCAATTG | 4 | 0/11 (0%) |
| GE2 | Off-target 2 | chr4: 128631982 | 3 | TTCCAcTTAAATGcATGGCAtTTG | 5 | 0/11 (0%) |
| GE3 | Off-target 1 | chr15: 91512242 | 3 | TTCCcGTTAAtTGGtTGGCAATTG | 6 | 0/11 (0%) |
| GE3 | Off-target 2 | chr4: 128631982 | 3 | TTCCAcTTAAATGcATGGCAtTTG | 7 | 0/11 (0%) |
| GE4 | Off-target 1 | chr15: 91512242 | 3 | TTCCcGTTAAtTGGtTGGCAATTG | 8 | 0/5 (0%) |
| GE4 | Off-target 2 | chr4: 128631982 | 3 | TTCCAcTTAAATGcATGGCAtTTG | 9 | 0/5 (0%) |

Amplification of the off-target candidate (predicted by CHOPCHOP v2) from the intended gene-edited iPSC clone was followed by direct sequencing around the candidate site by Sanger sequencing using a particular primer.
^{a}The underline indicates a PAM which can be recognized by an AsCpf1_RR mutant. The lowercase letters represent mismatch bases in the off-target candidates, as compared with the original target sequences.
^{b}The number of Indel clones relative to the number of analyzed clones.

The sequences of the primers and the like used in the present Examples are listed in the following Tables 3 to 5.

TABLE 3
Sequences of primers and the like (pY211puro construction)

| | | SEQ | | SEQ |
|---|---|---|---|---|
| | Forward | ID NO: | Reverse | ID NO: |
| HAx3 | GCAAAAAAGAAAAAGGGATCCTACCCATACGA | 10 | CCCTGCCCTCGCCGGAGCCGCTAGCGGCA | 11 |
| | TGTTCCAGATTACG | | TAGTCGGGGACATCATATG | |
| T2A | GGCTCCGGCGAGGGCAGGGGAAGTCTTTTGAC | 12 | TGGGCCGGGATTTTCCTCCACGTCCCCGCA | 13 |
| | ATGCGGGGACGTGGAGGAAAATCCCGGCCCA | | TGTCAAAAGACTTCCCCTGCCCTCGCCGGA | |
| | | | GCC | |
| PUROMYCIN | TCCCGGCCCAACTAGTACCGAGTACAAGCCCA | 14 | CGAGCTCTAGGAATTCTCAGGCACCGGGCT | 15 |
| resistance gene | CGGTG | | TGCGGGT | |
| amplification | | | | |

TABLE 4
Sequences of crRNA guide RNA templates, gene-editing templates, and the like

| | | SEQ | | SEQ |
|---|---|---|---|---|
| | Forward | ID NO: | Reverse | ID NO: |
| RET_M918 target site | GGGAAGCACTGCTCTGCACTAC | 16 | TGCTCAGGGCCAGTGCAATT | 17 |
| PCR amplification | | | | |
| RET_M918 target site | GTGTGTGGCCAGTTCTGTGC | 18 | | |
| sequencing | | | | |
| ssODN_RET_M918_I913_silentC | TTATTCCATCTTCTCTTTAGGGTCGGATCCCAGTTAAA | 19 | | |
| (WT) | TGGATGGCAATTGAATCCCTTTTTGATCATATCTACAC | | | |
| | CACGCAAAGTGATGTGTAAGTGT | | | |
| ssODN_RET_M918T_I913_silentC | TTATTCCATCTTCTCTTTAGGGTCGGATCCCAGTTAAA | 20 | | |
| (Mut) | TGGACGGCAATTGAATCCCTTTTTGATCATATCTACAC | | | |
| | CACGCAAAGTGATGTGTAAGTGT | | | |
| ssODN_RET_M918_I920_silentC | TTATTCCATCTTCTCTTTAGGGTCGGATTCCAGTTAAA | 21 | | |
| (WT) | TGGATGGCAATCGAATCCCTTTTTGATCATATCTACAC | | | |
| | CACGCAAAGTGATGTGTAAGTGT | | | |
| ssODN_RET_M918 | TTATTCCATCTTCTCTTTAGGGTCGGATTCCAGTTAAA | 22 | | |
| (WT) | TGGATGGCAATTGAATCCCTTTTTGATCATATCTACAC | | | |
| | CACGCAAAGTGATGTGTAAGTGT | | | |
| RET_I913 SNP-PCR | CCATCTTCTCTTTAGGGTCGGATT | 23 | ACACTTACACATCACTTTGCGTGG | 24 |
| primer WT | | | | |
| RET_I913 SNP-PCR | CCATCTTCTCTTTAGGGTCGGATC | 25 | ACACTTACACATCACTTTGCGTGG | 24 |
| primer Mut | | | | |
| RET_M918 SNP-PCR | GGGTCGGATTCCAGTTAAATGGAT | 26 | ACACTTACACATCACTTTGCGTGG | 24 |
| primer WT | | | | |
| RE_M918 SNP-PCR | GGGTCGGATTCCAGTTAAATGGAC | 27 | ACACTTACACATCACTTTGCGTGG | 24 |
| primer Mut | | | | |
| RET_I920 SNP-PCR | GGATTCCAGTTAAATGGATGGCAATC | 28 | ACACTTACACATCACTTTGCGTGG | 24 |
| primer WT | | | | |
| RET_I920 SNP-PCR | GATTCCAGTTAAATGGACGGCAATC | 29 | ACACTTACACATCACTTTGCGTGG | 24 |
| primer Mut | | | | |
| RET_M918 off-target1 | TTCATTTACAGGCGTACTTCG | 30 | ATGACTACCGGTTTCCCAAT | 31 |
| RET_M918 off-target2 | CGCCTGTAATCCCAGTTACT | 32 | AAAGAGCTATGGTTCCTTGCTCTG | 33 |
| RET_M918 off-target1 | ACTACCGCTTTCCCAATCAA | 34 | | |
| sequencing | | | | |
| RET_M918 off-target2 | GTTACTCAGGAGGCTGAGGC | 35 | | |
| sequencing | | | | |

TABLE 5
Sequences of crRNA guide RNA templates, gene-editing templates, and the like

| | | SEQ | | SEQ |
|---|---|---|---|---|
| | Forward | ID NO: | Reverse | ID NO: |
| COL7A1_G2138X target site | TCTGTGGATGAGCCAGGTCCTG | 36 | CCTTAGTTTCCCAGTTCCAACTTCC | 37 |
| PCR amplification | | | | |
| COL7A1_G2138X target site | GGTGACCAAGGTCCCAAAGG | 38 | | |
| sequencing | | | | |
| ssODN COL7A1_G2138 | CTTACCGGGTTGCCGTCCTGACCCCTCGGTCCAGG | 39 | | |
| (WT) | CTCTCCCCGGTCTCCTTTGATGCCTGGCACACCCT | | | |
| | GAAGGCAGAGTGTCGTGCCCTGAGCCCCC | | | |
| ssODN COL7A1_G2138X | CTTACCGGGTTGCCGTCCTGACCCCTCGGTCCAGG | 40 | | |
| (Mut) | CTCTCCCCGGTCTCATTTGATGCCTGGCACACCCT | | | |
| | GAAGGCAGAGTGTCGTGCCCTGAGCCCCC | | | |
| COL7A1_G2138 SNP-PCR | GGGTGTGCCAGGCATCAAAG | 41 | CTTACCGGGTTGCCGTCCT | 42 |
| primer WT | | | | |
| COL7A1_G2138 SNP-PCR | GGGTGTGCCAGGCATCAAAT | 43 | CTTACCGGGTTGCCGTCCT | 42 |
| primer Mut | | | | |

All the publications, patents, and patent applications cited herein are incorporated herein by reference in their entirety.

1. A method of producing a culture cell, comprising:
a) culturing a plurality of animal cells or plant cells in a culture vessel by adherent culture or on a semisolid medium to form a plurality of colonies, each of which being derived from a single cell;
b) detecting a sequence of one or more base(s) in a nucleic acid for a part of said plurality of colonies by a nucleic acid or protein detection method, during which said plurality of colonies are cultured in a culture vessel; and
c) selecting and collecting said part of the colonies based on the results of said detection method.

2. The method according to claim 1, comprising a step of further culturing the cells collected in said step c).

3. The method according to claim 1, wherein said gene or protein detection method is single-base mismatch detection PCR.

4. The method according to claim 1, wherein the cells are cultured by adherent culture in said step a).

5. The method according to claim 1, wherein said plurality of colonies are cultured for 4 hours to 6 days in said step b).

6. The method according to claim 1, wherein animal cells are cultured in said step a).

7. The method according to claim 6, wherein said animal cells are mammalian cells.

8. The method according to claim 7, wherein said mammalian cells are stem cells.

9. The method according to claim 1, wherein said culture vessel has an identifier whereby the position of each colony in the vessel can be identified, and wherein said part of the colonies are identified and selected based on said identifier in said step c).

10. The method according to claim 9, wherein said culture vessel having an identifier is a plate with grid.

11. The method according to claim 1, comprising a step of further subjecting said part of the plurality of colonies to another gene or protein detection method before or after said step c).

12. The method according to claim 1, further comprising a step of performing genome editing for said animal cells or plant cells before said step a).

13. The method according to claim 12, wherein said genome editing substitutes a mutant base sequence with a wild-type base sequence, or a wild-type base sequence with a mutant base sequence in the genomic DNA.

14. The method according to claim 13, wherein said mutant base sequence causes a disease.

15. The method according to claim 12, wherein said genome editing does not introduce a marker base, and wherein the absence of a nucleotide existing before substitution by the genome editing is detected in said step b).

16. The method according to claim 12, wherein said genome editing is performed using a protein selected from the group consisting of TALEN and a modified protein thereof, SpCas9 and a modified protein thereof, SaCas9 and a modified protein thereof, ScCas9 and a modified protein thereof, AsCpf1 and a modified protein thereof, LbCpf1 and a modified protein thereof, FnCpf1 and a modified protein thereof, MbCpf1 and a modified protein thereof, CBE and a modified protein or an analogue thereof, and ABE and a modified protein or an analogue thereof.

17. A method of producing a culture cell, comprising:
a) substituting a mutant base sequence with a wild-type base sequence or substituting a wild-type base sequence with a mutant base sequence in the genomic DNA in an animal cell or plant cell by genome editing without introducing a marker base;
b) detecting the absence of a nucleotide existing before substitution by the genome editing in the genome-edited cell; and
c) selecting and collecting a cell in which the absence of the nucleotide existing before substitution is detected.

18. The method according to claim 17, wherein a single-base mismatch detection PCR detects the absence of the nucleotide existing before substitution by said genome editing.

19. The method according to claim 17, wherein said genome editing is performed using a protein selected from the group consisting of TALEN and a modified protein thereof, SpCas9 and a modified protein thereof, SaCas9 and a modified protein thereof, ScCas9 and a modified protein thereof, AsCpf1 and a modified protein thereof, LbCpf1 and a modified protein thereof, FnCpf1 and a modified protein thereof, MbCpf1 and a modified protein thereof, CBE and a modified protein or an analogue thereof, and ABE and a modified protein or an analogue thereof.

20. A genome-edited cell obtained by the method according to claim 12.

21. A pharmaceutical composition comprising the cell according to claim 20.


SEQUENCE LISTING (43 sequences)

SEQ ID NO: 1 - DNA, 24 bp, Homo sapiens
ttccagttaaatggatggcaattg

SEQ ID NO: 2 - DNA, 24 bp, Homo sapiens
ttcccgttaattggttggcaattg

SEQ ID NO: 3 - DNA, 24 bp, Homo sapiens
ttccacttaaatgcatggcatttg

SEQ ID NO: 4 - DNA, 24 bp, Homo sapiens
ttcccgttaattggttggcaattg

SEQ ID NO: 5 - DNA, 24 bp, Homo sapiens
ttccacttaaatgcatggcatttg

SEQ ID NO: 6 - DNA, 24 bp, Homo sapiens
ttcccgttaattggttggcaattg

SEQ ID NO: 7 - DNA, 24 bp, Homo sapiens
ttccacttaaatgcatggcatttg

SEQ ID NO: 8 - DNA, 24 bp, Homo sapiens
ttcccgttaattggttggcaattg

SEQ ID NO: 9 - DNA, 24 bp, Homo sapiens
ttccacttaaatgcatggcatttg

SEQ ID NO: 10 - DNA, 46 bp, Artificial
  primer
gcaaaaaagaaaaagggatcctacccatacgatgttccagattacg

SEQ ID NO: 11 - DNA, 49 bp, Artificial
  primer
cccctgccctcgccggagccgctagcggcatagtcggggacatcatatg

SEQ ID NO: 12 - DNA, 63 bp, Artificial
  primer
ggctccggcgagggcaggggaagtcttttgacatgcggggacgtggaggaaaatcccggccca

SEQ ID NO: 13 - DNA, 63 bp, Artificial
  primer
tgggccgggattttcctccacgtccccgcatgtcaaaagacttcccctgccctcgccggagcc

SEQ ID NO: 14 - DNA, 37 bp, Artificial
  primer
tcccggcccaactagtaccgagtacaagcccacggtg

SEQ ID NO: 15 - DNA, 37 bp, Artificial
  primer
cgagctctaggaattctcaggcaccgggcttgcgggt

SEQ ID NO: 16 - DNA, 22 bp, Artificial
  primer
gggaagcactgctctgcactac

SEQ ID NO: 17 - DNA, 20 bp, Artificial
  primer
tgctcagggccagtgcaatt

SEQ ID NO: 18 - DNA, 20 bp, Artificial
  primer
gtgtgtggccagttctgtgc

SEQ ID NO: 19 - DNA, 99 bp, Artificial
  synthetic
ttattccatcttctctttagggtcggatcccagttaaatggatggcaattgaatccctttttgatcatatctacaccacgcaaagtgatgtgtaagtgt

SEQ ID NO: 20 - DNA, 99 bp, Artificial
  synthetic
ttattccatcttctctttagggtcggatcccagttaaatggacggcaattgaatccctttttgatcatatctacaccacgcaaagtgatgtgtaagtgt

SEQ ID NO: 21 - DNA, 99 bp, Artificial
  synthetic
ttattccatcttctctttagggtcggattccagttaaatggatggcaatcgaatccctttttgatcatatctacaccacgcaaagtgatgtgtaagtgt

SEQ ID NO: 22 - DNA, 99 bp, Artificial
  synthetic
ttattccatcttctctttagggtcggattccagttaaatggatggcaattgaatccctttttgatcatatctacaccacgcaaagtgatgtgtaagtgt

SEQ ID NO: 23 - DNA, 24 bp, Artificial
  primer
ccatcttctctttagggtcggatt

SEQ ID NO: 24 - DNA, 24 bp, Artificial
  primer
acacttacacatcactttgcgtgg

SEQ ID NO: 25 - DNA, 24 bp, Artificial
  primer
ccatcttctctttagggtcggatc

SEQ ID NO: 26 - DNA, 24 bp, Artificial
  primer
gggtcggattccagttaaatggat

SEQ ID NO: 27 - DNA, 24 bp, Artificial
  primer
gggtcggattccagttaaatggac

SEQ ID NO: 28 - DNA, 26 bp, Artificial
  primer
ggattccagttaaatggatggcaatc

SEQ ID NO: 29 - DNA, 25 bp, Artificial
  primer
gattccagttaaatggacggcaatc

SEQ ID NO: 30 - DNA, 21 bp, Artificial
  primer
ttcatttacaggcgtacttcg

SEQ ID NO: 31 - DNA, 20 bp, Artificial
  primer
atgactaccggtttcccaat

SEQ ID NO: 32 - DNA, 20 bp, Artificial
  primer
cgcctgtaatcccagttact

SEQ ID NO: 33 - DNA, 24 bp, Artificial
  primer
aaagagctatggttccttgctctg

SEQ ID NO: 34 - DNA, 20 bp, Artificial
  primer
actaccggtttcccaatgaa

SEQ ID NO: 35 - DNA, 20 bp, Artificial
  primer
gttactcaggaggctgaggc

SEQ ID NO: 36 - DNA, 22 bp, Artificial
  primer
tctgtggatgagccaggtcctg

SEQ ID NO: 37 - DNA, 25 bp, Artificial
  primer
ccttagtttcccagttccaacttcc

SEQ ID NO: 38 - DNA, 20 bp, Artificial
  primer
ggtgaccaaggtcccaaagg

SEQ ID NO: 39 - DNA, 99 bp, Artificial
  synthetic
cttaccgggttgccgtcctgacccctcggtccaggctctccccggtctcctttgatgcctggcacaccctgaaggcagagtgtcgtgccctgagccccc

SEQ ID NO: 40 - DNA, 99 bp, Artificial
  synthetic
cttaccgggttgccgtcctgacccctcggtccaggctctccccggtctcatttgatgcctggcacaccctgaaggcagagtgtcgtgccctgagccccc

SEQ ID NO: 41 - DNA, 20 bp, Artificial
  primer
gggtgtgccaggcatcaaag

SEQ ID NO: 42 - DNA, 19 bp, Artificial
  primer
cttaccgggttgccgtcct

SEQ ID NO: 43 - DNA, 20 bp, Artificial
  primer
gggtgtgccaggcatcaaat